(19) United States
(12) Patent Application Publication
(10) Pub. No.: US 2014/0018424 A1
(43) Pub. Date: Jan. 16, 2014

(54) PREVENTIVE EFFECT OF CITRULLINE ON THE SPONTANEOUS DEVELOPMENT OF TUMORS

(71) Applicants: Christophe Moinard, Bourg-Ia-reine (FR); Servane Le Plenier, Beynes (FR); Agathe Raynaud-Simon, Vincennes (FR); Vickie Baracos, Alberta (CA); Luc Cynober, Sceaux (FR)

(72) Inventors: Christophe Moinard, Bourg-Ia-reine (FR); Servane Le Plenier, Beynes (FR); Agathe Raynaud-Simon, Vincennes (FR); Vickie Baracos, Alberta (CA); Luc Cynober, Sceaux (FR)

(73) Assignee: UNIVERSITE PARIS DESCARTES, Paris Cedex 06 (FR)

(21) Appl. No.: 13/979,681

(22) PCT Filed: Jan. 10, 2012
(86) PCT No.: PCT/FR12/50068

§ 371 Date: Oct. 4, 2013

(30) Foreign Application Priority Data
Jan. 14, 2011 (FR) 11/50311

(51) Int. Cl. A61K 31/197

(52) U.S. Cl. CPC A61K 31/197
USPC 514/563; 562/560

(57) ABSTRACT

L-citrulline or a nutraceutically or pharmaceutically acceptable salt thereof for reducing the kinetics of tumour development or reduce the incidence of tumours in aged humans and animals.

A subject of the present invention is a novel use of citrulline.

Citrulline (or 2-amino-5-(carbamoyl amino)pentanoic acid) is an α-amino acid and was first isolated from watermelon. Citrulline is a non-essential amino acid that the body produces (in the intestine) from other nutrients. For example, citrulline plays a particularly important role, with ornithine and arginine, in the urea cycle. Finally, citrulline plays a major role in the homeostasis of arginine and free-radical nitric oxide.

Certain pharmaceutical uses of citrulline have already been described.

Thus, the European patent application EP 1495755 relates to the use of citrulline for the preparation of a medicament intended for the treatment of pathologies linked to intestinal failure. More particularly, the pathologies mentioned in this application are the following: short bowel syndrome following intestinal resection, coeliac disease, chronic inflammatory diseases of the intestine, intestinal failure linked to ageing and intestinal failure linked to radiation. The European patent application EP1755582 describes in particular the use of the combination of at least one statin with citrulline for the preparation of a medicament intended for the prevention or treatment of atherosclerosis, in particular of primary or secondary atherosclerosis, hypertension, diabetes or neurodegenerative diseases such as Alzheimer's disease. The patent FR2937550 describes the use of citrulline in the treatment and prevention of ischaemia-reperfusion syndrome. The application US 2009/02911877 describes the use of citrulline in the treatment of satiety and dyspepsia in particular in the elderly or in cancer patients. The application WO 2008/049984 describes the use of citrulline in the treatment of undernutrition conditions in cancer patients. The application WO 2007/114903 describes the use of L-citrulline for the treatment of digestive disorders such as the feeling of satiety and dyspepsia, in particular in cancer patients.

It is now generally accepted that the incidence of most cancers increases with advancing age. Thus in France, 5% of over-50s report having cancer. If breast, colon cancer, etc. are diseases that primarily affect the elderly, this is because a set of mutations and events contributes to their formation, for example, changes in hormone levels or repeated exposure to carcinogens originating from food or from the environment.

In fact, although genetic factors are heavily implicated in the development of cancers, environmental, in particular nutritional, factors also play a significant role. The occurrence of cancers is particularly widespread in developed countries and is linked to the use of pesticides and insecticides, to residual quantities of these in foods, to the consumption of additives such as preservatives and colouring agents, to water, soil and air pollution, to obesity caused by diets high in saturated fats, and to longer life which contributes to repeated exposure to carcinogens and to changes in the cell cycle.

The various cancer treatment procedures are mainly surgical treatment, chemotherapy, immunotherapy and radiotherapy. The surgical treatment procedure is effective in the early stages, but leads to the removal of the diseased organ, which can cause harmful secondary pathologies, and does not always prevent the propagation of the cancer to other organs. Radiotherapy is advantageous since it allows a more selective treatment of the diseased organ, but still does not prevent the cancer from spreading to other organs. Chemotherapy is known to act not only on the cancer cells but also on a patient's normal cells, sometimes causing disabling side effects. Thus, the anticancer treatments will have a major impact on the digestive system (which will result in the occurrence of nausea, vomiting and the development of anorexia and life-threatening cachexia).

Thus, despite the significant successes already achieved in the fight against cancer, the prevention and treatment of this disease are not generally satisfactory. As a result, it remains necessary to develop more effective means of treating cancer and above all, so far as possible, to try to prevent it. In fact, all of the anticancer strategies developed have side effects which are very disabling and which can be life-threatening (by way of example, the mTOR inhibitors will exacerbate the problem of cachexia).

The patent U.S. Pat. No. 4,988,724 describes parenteral solutions capable of preventing the stimulation of tumour growth in cancer patients, said solutions containing a mixture of essential amino acids, ornithine and citrulline, of non-essential acids amines and being practically devoid of arginine. The composition of these solutions is based on the discovery that arginine, a polyamine precursor, contained in the parenteral solutions commonly used in cancer patients, was responsible for tumour growth. As a result, the inventors propose replacing the arginine with ornithine. In these compositions, citrulline is used to replace all or part of the ornithine as urea cycle substrate.

The inventors have surprisingly discovered that the long-term administration of citrulline to aged healthy mammals reduces mortality and lowers the incidence of cancer tumour development.

Thus, one of the aims of the present invention is to provide a means for preventing or treating cancers in aged healthy mammals.

Consequently, a subject of the present invention is the use of L-citrulline, or a nutraceutically or pharmaceutically acceptable salt thereof, to reduce the kinetics of tumour development or to reduce the incidence of tumours in humans over the age of 70 and in aged animals.

Within the meaning of the present invention, by aged animal is meant 10 years in dogs, 13 years in cats, 20 months in rodents and 1 year in fish.

In the present invention, by “pharmaceutically acceptable salt” is meant in particular citrulline salts such as citrulline malate, citrulline α-ketoglutarate, citrulline citrate or citrulline α-ketoisocaproate.

Within the meaning of the present invention, by L-citrulline is meant the commercially available product, in particular supplied by Sigma-Aldrich, Biocodex or Kyowa Hakko or the natural product originating from plants, in particular from watermelon (Citrullus lanatus) particularly in the form of juice, pulp or extract.

In an advantageous embodiment of the invention, L-citrulline can be presented in the form of a food composition in combination with a nutraceutically acceptable excipient or in the form of a pharmaceutical composition in combination with a pharmaceutically acceptable excipient.

According to the invention, the cancer is chosen from the group comprising: cancers of the head or neck, lung, gastrointestinal and pleural cancers, cancers of the liver, kidney, pancreas or immune system (leukaemia).

In an advantageous embodiment of the invention, L-citrulline can be combined with a compound chosen from the group comprising glutamine, the polyunsaturated fatty acids (PUFAs), antioxidants such as for example vitamins C, E and resveratrol), zinc, selenium or anticancer micronutrients (such as for example platinum salts, 5-fluorouracil or methotrexate) algae, nutrients such as curcumin, glucosinolate or the polyphenols.

The dosage of citrulline depends particularly on the administration method, and is easily determined by a person skilled in the art. For example, without however being limitative, the compositions as used in the invention are capable of being administered by oral route at a dose of 5 to 20 g/day for a period of from 1 day to 10 years, advantageously from 1 to 5 years, by parenteral route at a dose of from 1 to 10 g for a period of 1 month to 10 years, in particular within the context of long-term nutrition at home, advantageously from 6 month to 5 years (the same doses can be used by oral, enteral or parenteral route, with different administration procedures).

The compositions used according to the invention can be presented in any form suitable for oral, subcutaneous, intravenous or enteral administration in particular; they can be presented in dry form, in the form of aqueous solution or in the form of emulsion.

A subject of the present invention is also the use of L-citrulline or a pharmaceutically acceptable salt thereof for the preparation of a medicament intended for reducing the kinetics of tumour development or reducing the incidence of tumours in humans over the age of 70 and aged animals.

A subject of the present invention is also the use of L-citrulline or a pharmaceutically acceptable salt thereof for the preparation of a composition intended for reducing the kinetics of tumour development or reducing the incidence of tumours in humans and animals.

A subject of the present invention is also the use of L-citrulline or a nutraceutically acceptable salt thereof for the preparation of a food supplement or of a dietetic product (dietary foods for special medical purposes) intended for reducing the kinetics of tumour development or reducing the incidence of tumours in humans and animals.

Within the meaning of the present invention, by “dietary foods for special medical purposes” is meant foods intended to meet the nutritional needs of patients whose metabolism is disturbed and who are unable to correctly assimilate the nutrients originating from conventional food.

A subject of the present invention is also a method making it possible to reduce the kinetics of tumour development or to reduce the incidence of tumours in humans and animals, comprising the administration to said mammal of a therapeutically effective quantity of L-citrulline.

The invention is illustrated by the following example.

Long-Term Effect of Citrulline in Aged Healthy Rats

1. Material and Methods

Thirty-nine male Sprague-Dawley rats aged 20 months and free of tumours (Charles River, L'Arbresle, France) are used. They are raised individually in cages, at a temperature of 20-23° C., subjected to a cycle of 12 hours of light alternating with 12 hours of darkness. They have free access to water. For the acclimatization period, all the rats are fed a standard diet (59% carbohydrates, 3% lipids, 17% proteins and 21% fibres) ad libitum (UAR of 0.4 Dietex, Villemoisson-sur-Orge, France) for 2 weeks. The animals are cared for in accordance with French regulations on the protection of animals used for experimental and scientific purposes (D 2001-486) and with the Regulations of the European Community (Official Journal of the European Community, L538 12:18:1986).

After the acclimatization period, the rats are randomly divided into 2 groups.

In the “citrulline” group (CIT, n=19), the rats are fed ad libitum for 12 weeks with a citrulline-based supplement (1 g.kg⁻¹.day⁻¹ of citrulline) mixed with their standard diet. The dose of citrulline is calculated by extrapolation of the doses used in humans, taking into account the fact that the metabolic rate and nitrogen requirements are 10 times greater in rodents than those measured in humans and very close to the dose of arginine administered to rats for 12 weeks by Jobgen W. et al. (J Nutr 2009;139:230-7). This dose corresponds to approximately 9% of the nitrogen intake, i.e. approximately 7g/day in the healthy adult volunteer.

In the “non-essential amino acids” group (NEAA, n=20), the rats are fed ad libitum for 12 weeks with a standard food rendered isonitrogenous with respect to the citrulline group by the addition of non-essential amino acids: histidine, serine, alanine and glycine in an equimolar ratio.

The rats are euthanized in the post-absorptive state after treatment for 12 weeks. The statistical studies are carried out using Statview® software

2. Results

### Mortality

The mortality over 12 weeks is 20% (4/20) in the NEAA group (which is consistent with the data in the literature for this species and at this age) whereas no mortality is observe in the CIT group (0/19) (NEAA vs CIT, p<0.05)

### Incidence of Tumours

After euthanasia, the incidence of the tumours is assessed in the two groups. In the NEAA group 44% of the rats (7/16) exhibit tumours, values consistent with what is described in the literature. On the other hand, in the CIT group none of the rats exhibits tumours (0/19) (NEAA vs CIT, p<0.05).
   Body weight
Supplementation with citrulline does not affect the body weight as shown by the results compiled in the table below:

| | NEAA | CIT |
|---|---|---|
| Initial body weight | 711 ± 21 | 669 ± 16* |
| Final body weight | 658 ± 15 | 630 ± 13** |

*NEAA vs CIT p = N.S
**NEAA vs CIT p = N.S

Level of polyamines

Supplementation with citrulline induces a significant increase in a polyamine, putrescine, as shown by the table below:

| | Citrulline group | AANE group | |
|---|---|---|---|
| | (n = 19) | (n = 16) | p |
| Liver putrescine | 17.1 ± 14.5 | 7.8 ± 5.1 | 0.034 |
| Liver spermidine | 372 ± 90 | 402 ± 119 | NS |
| Liver spermine | 557 ± 245 | 557 ± 353 | NS |
| Jejunum putrescine | 82.7 ± 36 | 82.8 ± 44.9 | NS |
| Jejunum spermidine | 478 ± 200 | 429 ± 131 | NS |
| Jejunum spermine | 297 ± 88 | 293 ± 74 | NS |
| Ileum putrescine | 79.8 ± 25 | 59.3 ± 34.6 | 0.028 |
| Ileum spermidine | 374 ± 70 | 326 ± 96 | NS |
| Ileum spermine | 203 ± 43 | 182 ± 48 | NS |

These results clearly indicate that, despite the increase in putrescine content observed after administration of citrulline, surprisingly, the administration of citrulline reduces mortality and the incidence of tumours in aged rats even though the polyamines are known to promote tumour growth and proliferation.

1. L-citrulline or a nutraceutically or pharmaceutically acceptable salt thereof for use in order to reduce the kinetics of tumour development or to reduce the incidence of tumours in humans over the age of 70 and in aged animals.

2. L-citrulline according to claim 1 characterized in that it is presented in the form of a food composition in combination with a nutraceutically acceptable excipient or in the form of a pharmaceutical composition in combination with a pharmaceutically acceptable excipient.

3. L-citrulline according to claim 1 characterized in that the cancer is chosen from the group comprising: cancers of the head or neck, lung, gastrointestinal and pleural cancers, cancers of the liver, kidney, pancreas or immune system (leukaemia).

4. L-citrulline according to claim 1 characterized in that it is combined with a compound chosen from the group comprising glutamine, the polyunsaturated fatty acids, antioxidants, micronutrients and anticancer agents.

5. L-citrulline according to claim 1 characterized in that the compositions have a dosage allowing the administration of a dose of from 5 to 20 g/day for a period of from 1 day to 10 years, advantageously from 1 to 5 years.

6. L-citrulline according to claim 1 characterized in that the composition is presented in dry form, in the form of aqueous solution or in the form of emulsion.

7. L-citrulline according to claim 1 characterized in that the composition can be administered by oral, subcutaneous, intravenous or enteral route.

8. A method for reducing the kinetics of tumour development or reducing the incidence of tumours in humans and animals, comprising administering an effective amount of L-citrulline or a pharmaceutically acceptable salt thereof to a human or animal in need thereof.

9. A composition comprising L-citrulline or a pharmaceutically acceptable salt thereof.

10. A food supplement or a dietetic product (dietary foods for special medical purposes) comprising L-citrulline or a nutraceutically acceptable salt thereof.

11. A method for reducing the kinetics of tumour development or reducing the incidence of tumours in humans and animals, comprising administering an effective amount of the composition according to claim 9 to a human or animal in need thereof.

12. A method for reducing the kinetics of tumour development or reducing the incidence of tumours in humans and animals, comprising administering an effective amount of the food supplement or a dietetic product (dietary foods for special medical purposes) according to claim 10 to a human or animal in need thereof.
